(19) United States
(12) Patent Application Publication
(10) Pub. No.: US 2016/0130291 A1
(43) Pub. Date: May 12, 2016

(54) PLATINUM COMPLEXES AS ANTICANCER AGENTS

(71) Applicant: King Fahd University of Petroleum and Minerals, Dhahran (SA)

(72) Inventors: Ahmed Zainelabdeen Abdalla Mustafa, Dhahran (SA); Muhammad Monim-Ul-Mehboob, Dhahran (SA); Muhammad Altaf, Dhahran (SA); Anvarhusein Abdulkadir Isab, Dhahran (SA)

(73) Assignee: King Fahd University of Petroleum and Minerals, Dhahran (SA)

(21) Appl. No.: 14/539,868

(22) Filed: Nov. 12, 2014

(51) Int. Cl. C07F 15/00; A61K 31/506

(52) U.S. Cl. CPC C07F 15/0093; A61K 31/506

(57) ABSTRACT

Compounds comprising a Pt(II) metal center and S-containing ligands in accordance to Formula I as anticancer agents. Methods of treating cancer comprising administering the compounds are also provided.

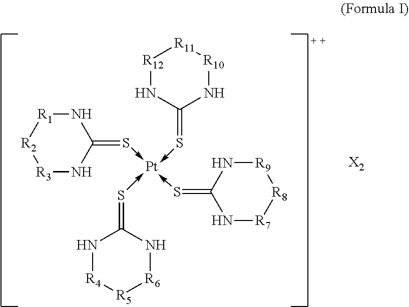

### BACKGROUND OF THE INVENTION

1. Technical Field

The present invention relates to compounds and methods of treating cancer. More specifically, the present invention relates to platinum-based complexes for use in chemotherapy.

2. Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

The use of cisplatin, oxaliplatin and carboplatin anticancer agents is well-known and has been well-documented in the literature (B. Rosenberg, L. Van Camp, T. Krigas, Inhibition of cell division in Escherichia coli by electrolysis products from a platinum electrode, Nature 205 (1965) 698-699; L. B. Travis, R. E. Curtis, H. Storm, P. Hall, E. Holowaty, F. E. Van Leeuwen, B. A. Kohler, E. Pukkala, C. F. Lynch, M. Andersson, K. Bergfeldt, E. A. Clarke, T. Wiklund, G. Stoter, M. Gospodarowicz, J. Sturgeon, J. F. Fraumeni, J. D. Boice, Risk of second malignant neoplasms among long-term survivors of testicular cancer, J. Natl. Cancer Inst. 8 (1997) 1429-1439; J. Reedijk, Why does cisplatin reach guanine-N7 with competing S-donor ligands available in the cell? Chem. Rev. 99 (1999) 2499-2510; E. Barreiro, J. S. Casas, M. D. Couce, A. Sanchez, J. Sordo, E. M. Vazquez-Lopez, Heteronuclear gold(I)-silver(I) sulfanylcarboxylates: synthesis, structure and cytotoxic activity against cancer cell lines, J. Inorg. Biochem. 131 (2014) 68-75; E. Ferrari, R. Grandi, S. Lazzari, G. Marverti, M. C. Rossi, M. Saladini, ¹H, ¹³C, ¹⁹⁵Pt NMR study on platinum(II) interaction with sulphur containing Amadori compounds, Polyhedron 26 (2007) 4045-4052.—each incorporated herein by reference in its entirety). These drugs have been used for the chemotherapy of cancer patients all over the world and hundreds of thousand patients have benefited from them. However, the major side effects of chemotherapy of cancer treatment with platinum based drugs are neurotoxicity and occurrence of resistance (S. Ahmad, A. Isab, S. Ali, Structural and mechanistic aspects of platinum anticancer, Agents, Transit. Met. Chem. 31 (2006) 1003-1016; S. R. McWhinney, R. M. Goldberg, H. L. McLeod, Platinum neurotoxicity pharmacogenetics, Mol. Cancer Ther. 8 (2009) 10-16; D. J. Stewart, Mechanism of resistance to cisplatin and carboplatin, Crit. Rev. Oncol. Hematol. 63 (2007) 12-31—each incorporated herein by reference in its entirety). During chemotherapy, DNA is the ultimate target for platinum based drugs but many sulfur containing biomolecules also interact with platinum drugs, especially thioether and thiols (K. R. Barnes, S. J. Lippard, J. Stephen, Cisplatin and related anticancer drugs: recent advances and insights, Met. Ions Biol. Syst. 42 (2004) 143-177; C. Bischin, A. Lupan, V. Taciuc, R. Silaghi-Dumitrescu, Interactions between proteins and platinum-containing anti-cancer drugs, Mini-Rev. Med. Chem. 11 (2011) 214-224—each incorporated herein by reference in its entirety). The sulfur containing biomolecules like GSH (glutathione) are present in very low concentration in many cells; the platinum drugs approach these biomolecules and make Pt\GSH bond, which plays a very important role in the anticancer activity of these drugs (M. M. Jennerwein, A. Eastman, A polymerase chain reaction-based method to detect cisplatin adducts in specific genes, Nucleic Acids Res. 19 (1999) 6209-6224; A. J. Jansen, J. Brower, J. Reedijk, Glutathione induces cellular resistance against cationic dinuclear platinum anticancer drugs, J. Inorg. Biochem. 89 (2002) 197-202—each incorporated herein by reference in its entirety). It has been observed that platinum drug resistant cells have elevated GSH levels (S. J. Berners-Price, P. W. Kuchel, Reaction of cis- and trans-[PtCl₂(NH₃)₂] with reduced glutathione studied by ¹H, ¹³C, ¹⁹⁵Pt and ¹⁵N-{¹H} DEPT NMR, J. Inorg. Biochem. 38(1990) 305-326—each incorporated herein by reference in its entirety). This type of side effects can be reduced by reversal or prevention of Pt\S adducts in the proteins (R. T. Dorr, Platinum and other metal coordination compounds in cancer chemotherapy vol. 2, Plenum Press, New York, 1996—incorporated herein by reference in its entirety). With this knowledge, new metal complexes with S donor atom ligand have been studied and some of them show promising cytotoxic and anticancer activities (R. del Campo, J. J. Criado, E. Garcia, M. R. Hermosa, A. Jimenez-Sanchez, J. L. Manzano, E. Monte, E. Rodriguez-Fernandez, F. Sanz, Thiourea derivatives and their nickel(II) and platinum(II) complexes: antifungal activity, J. Inorg. Biochem. 89 (2002) 74-82; B. A. Al-Maythalony, M. Monim-ul-Mehboob, M. Altaf, M. I. M. Wazeer, A. A. Isab, S. Altuwaijri, A. Ahmed, V. Dhuna, G. Bhatia, K. Dhuna, S. S. Kamboj, Some new [(thione)2Au(diamine)]Cl3 complexes: synthesis, spectroscopic characterization, computational and in vitro cytotoxic studies, Spectrochim. Acta A 115 (2013) 641-647; J. Lin, G. Lu, L. M. Daniels, X. Wei, J. B. Sapp, Y. Deng, Synthesis and characterization of platinum(II) complexes with 2-imidazolidinethione. X-ray crystal structure of tetra(2-imidazolidinethione-S)platinum(II) iodide dimethylsulfoxide solvate monohydrate, J. Coord. Chem. 61 (2008) 2457-2469; M. Altaf, H. Stoeckli-Evans, A. Cuin, D. N. Sato, F. R. Pavan, Q. F. Leite, S. Ahmad, M. Bouakka, M. Mimouni, F. Z. Khardli, T. B. Hadda, Synthesis, crystal structures, antimicrobial, antifungal and antituberculosis activities of mixed ligand silver(I) complexes, Polyhedron 62 (2013) 138-147; C. Vetter, C. Wagner, J. Schmidt, D. Steinborn, Synthesis and characterization of platinum(IV) complexes with N—S and S— S heterocyclic ligands, Inorg. Chim. Acta 359 (2006) 4326-4334; E. Khazanov, Y. Barenholz, D. Gibson, Y. Najajreh, Novel apoptosis-inducing transplatinum piperidine derivatives: synthesis and biological characterization, J. Med. Chem. 45 (2002) 5196-5204; A. G. Quiroga, Understanding the trans platinum complexes as potential antitumor drugs beyond targeting DNA, J. Inorg. Biochem. 114 (2012) 106-112; G. Natile, M. Coluccia, Current status of trans-platinum compounds in cancer therapy, Coord. Chem. Rev. 216-217 (2001) 383-410; Y. Najajreh, J. M. Perez, C. Navarro-Ranninger, D. Gibson, Novel soluble cationic trans-diaminedichloroplatinum(II) complexes that are active against cisplatin resistant ovarian cancer cell lines, J. Med. Chem. 45 (2002) 5189-5195; G. Cervantes, S. Marchal, M. J. Prieto, J. M. Perez, V. M. Gonzalez, C. Alonso, V. Moreno, DNA interaction and antitumor activity of a Pt(III) derivative of 2-mercaptopyridine, J. Inorg. Chem. 77 (1999) 197-203; M. Mizota, Y. Yokoyama, K. Sakai, Tetrakis[pyridine-2(1H)-thione-κS]platinum(II) dichloride, Acta Cryst. (2005) 1433-1435; T. Lobana; R. Verma, G. Hundal, A. Castineiras, Metal-heterocyclic thione interactions: 12. Heterocyclic 2-thiolates of platinum)II) and palladium(II): the crystal structure of first examples of cis-[M(η-S-pyridine-2-thiolato)2(L-L)] (M=Pt, Pd, L-L=1,2-bis(diphenylphosphino)ethane; M=Pt, L-L=1,2-bis(diphenylphosphinoethene) complexes, Polyhedron 19 (2000) 899-906; W02002020027A1; CN103554188A—each incorporated herein by reference in its entirety).

Platinum containing complexes synthesized with S containing ligands like heterocyclic and aliphatic thiones have been shown good cytotoxic and anticancer activities; sometimes higher than that of cisplatin (E. Khazanov, Y. Barenholz, D. Gibson, Y. Najajreh, Novel apoptosis-inducing transplatinum piperidine derivatives: synthesis and biological characterization, J. Med. Chem. 45 (2002) 5196-5204; A. G. Quiroga, Understanding the trans platinum complexes as potential antitumor drugs beyond targeting DNA, J. Inorg. Biochem. 114 (2012) 106-112; G. Natile, M. Coluccia, Current status of trans-platinum compounds in cancer therapy, Coord. Chem. Rev. 216-217 (2001) 383-410; Y. Najajreh, J. M. Perez, C. Navarro-Ranninger, D. Gibson, Novel soluble cationic trans-diaminedichloroplatinum(II) complexes that are active against cisplatin resistant ovarian cancer cell lines, J. Med. Chem. 45 (2002) 5189-5195—each incorporated herein by reference in its entirety). Therefore, these types of complexes are considered as potential anticancer agents (G. Cervantes, S. Marchal, M. J. Prieto, J. M. Perez, V. M. Gonzalez, C. Alonso, V. Moreno, DNA interaction and antitumor activity of a Pt(III) derivative of 2-mercaptopyridine, J. Inorg. Chem. 77 (1999) 197-203; M. Carrara, T. Berti, S. D'Ancona, V. Cherchi, L. Sindellari, In vitro effect of Pt and Pd mercaptopyridine complexes, Anticancer Res. 17 (1997) 975-980; G. Cervantes, M. J. Prieto, V. Moreno, Antitumor activity of a Pt(III) derivative of 2-mercaptopyrimidine, Met.-Based Drugs 4 (1997) 9-18.—each incorporated herein by reference in its entirety).

In the present invention, focus is given on design and exploration of precious metal complexes as anticancer agents M. Monim-ul-Mehboob, M. Altaf, M. Fettouhi, A. A. Isab, M. I. M. Wazeer, M. N. Shaikh, S. Altuwaijri, Synthesis, spectroscopic characterization and anti-cancer; properties of new gold(III)—alkanediamine complexes against gastric, prostate and ovarian cancer cells; crystal structure of [Au2(pn)2(Cl)2]Cl2.H2O, Polyhedron 61 (2013) 225-234; S. S. Al-Jaroudi, M. Fettouhi, M. I. M. Wazeer, A. A. Isab, S. Altuwaijri, Synthesis, characterization and cytotoxicity of new gold(III) complexes with 1,2-diaminocyclohexane: Influence of stereochemistry on antitumor activity, Polyhedron 50 (2013) 434-442; A. A. Isab, M. N. Shaikh, M. Monim-ul-Mehboob, B. A. Al-Maythalony, M. I. M. Wazeer, S. Altuwaijri, Synthesis, characterization and anti-proliferative effect of [Au(en)₂]Cl₃ and [Au(N-propyl-en)₂]Cl₃ on human cancer cell lines, Spectrochim. Acta-Part A 79 (2011) 1196-1201—each incorporated herein by reference in its entirety).

Accordingly, a need exists for novel platinum-based anticancer agents with improved cytotoxic properties and reduced neurotoxicity and occurrence of resistance.

### BRIEF SUMMARY OF THE INVENTION

The foregoing paragraphs have been provided by way of general introduction, and are not intended to limit the scope of the following claims. The described embodiments, together with further advantages, will be best understood by reference to the following detailed description taken in conjunction with the accompanying drawings.

In a first aspect, the present invention relates to novel compounds with a Pt(II) metal center and a plurality of S-containing ligands as anticancer agents, wherein said compounds having a general formula as the following Formula I:

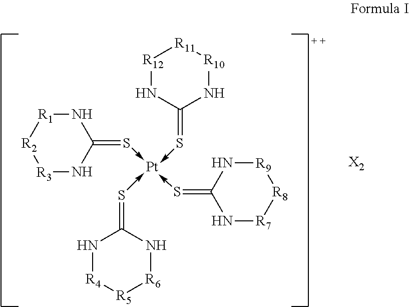

or a pharmaceutically acceptable salt, ester or prodrug thereof;

wherein:

X is selected from hydroxide, hydride, fluoride, chloride, bromide, iodide, cyanide, amide, cyanate, thiocyanate, permanganate, acetate, formate, nitrite, nitrate, hydrogen sulfate, dihydrogen phosphate, perchlorate, iodate, chlorate, bromate, chlorite, hypochlorite, hypobromite, bicarbonate or other pharmaceutically acceptable anion; and

R₁, R₂, R₃, R₄, R₅, R₆, R₇, R₈, R₉, R₁₀, R₁₁ and R₁₂ are radicals, each independently selected from substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl, preferably —CH₂—, —CH₂CH₃—.

In a preferred embodiment, the compound is tetrakis(1,3-diazinane-2-thione)platinum(II) chloride monohydrate complex ([Pt(Diaz)₄]Cl₂.H₂O, wherein Diaz=1,3-diazinane-2-thione), referred to as compound 1 throughout the present disclosure having the following Formula II:

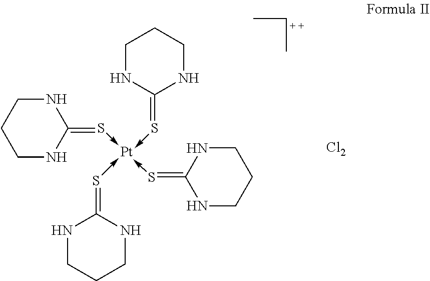

or a pharmaceutically acceptable salt, ester or prodrug thereof.

In a second aspect, a pharmaceutical composition comprising the compound having Formula II as shown above is provided. The pharmaceutical composition may further comprise other active pharmaceutical agents, non-active ingredients, a pharmaceutically acceptable carrier in solid, semi-solid or liquid forms. The pharmaceutical composition may be administered to the subject in need thereof systemically, parenterally, intramuscularly, intraperitoneally, transdermally, extracorporeally, topically, or any combination thereof.

In a third aspect, a method of treating cancer comprising administering to a subject in need thereof an effective amount of a pharmaceutical composition comprising the compound having Formula I is also provided. The pharmaceutical composition may further comprise other active pharmaceutical agents, non-active ingredients, a pharmaceutically acceptable carrier in solid, semi-solid or liquid forms. The pharmaceutical composition may be administered to the subject in need thereof systemically, parenterally, intramuscularly, intraperitoneally, transdermally, extracorporeally, topically, or any combination thereof. The disclosed method is effective in treating, for example, lung (small and non-small cell), breast, colorectal, cervical, ovarian, testicular, pancreatic, gastric, liver, bladder, head and neck, brain, oral cancers, leukemia, gliomas, glioblastomas, sarcomas, carcinomas, lymphomas, myelomas, germ cell tumors.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is an ORTEP diagram of compound 1 showing the atomic labeling scheme.

FIG. 2 is a molecular packing diagram of compound 1 showing the hydrogen bonding patterns.

FIG. 3 is a ¹⁹⁵Pt CPMAS spectrum of compound 1 at a spinning rate of 11 kHz with the isotropic peak marked with an asterisk.

### DETAILED DESCRIPTION OF THE EMBODIMENTS

Referring now to the drawings, wherein like reference numerals designate identical or corresponding parts throughout the several views.

As used herein, the terms “compound” and “complex” are intended to refer to a chemical entity, whether in the solid, liquid or gaseous phase, and whether in a crude mixture or purified and isolated.

The term “alkyl”, as used herein, unless otherwise specified, refers to a saturated straight, branched, or cyclic, primary, secondary, or tertiary hydrocarbon of typically C₁ to C₁₂, and specifically includes methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term optionally includes substituted alkyl groups. Moieties with which the alkyl group can be substituted are selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis”, John Wiley and Sons, Second Edition, 1991, hereby incorporated by reference in its entirety.

The term “alkenyl” means a monovalent, unbranched or branched hydrocarbon chain having one or more double bonds therein. The double bond of an alkenyl group can be unconjugated or conjugated to another unsaturated group. Suitable alkenyl groups include, but are not limited to (C₂-C₁₂)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-ethenyl)-pentenyl. An alkenyl group can be unsubstituted or substituted with one or more suitable substituents.

The term “alkynyl” means a monovalent, unbranched or branched hydrocarbon chain having one or more triple bonds therein. The triple bond of an alkynyl group can be unconjugated or conjugated to another unsaturated group. Suitable alkynyl groups include, but are not limited to (C₂-C₁₂)alkynyl groups, such as ethynyl, propynyl, butyryl, pentynyl, hexynyl, 2-ethylhexynyl, 2-propyl-2-butyryl, 4-(2-methyl-3-ethynyl)-pentynyl. An alkynyl group can be unsubstituted or substituted with one or more suitable substituents.

The term “aryl”, as used herein, and unless otherwise specified, refers to phenyl, biphenyl, or naphthyl, and preferably phenyl. The term includes both substituted and unsubstituted moieties. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis”, John Wiley and Sons, Second Edition, 1991, hereby incorporated by reference in its entirety.

The term “halo”, as used herein, includes chloro, bromo, iodo, and fluoro.

The term “haloalkyl” refers an alkyl group which is substituted by at least one halo group, for example CF₃.

The term “pharmaceutically acceptable salt, ester or prodrug” is used throughout the specification to describe any pharmaceutically acceptable form (such as an ester, phosphate ester, salt of an ester or a related group) of a compound which, upon administration to a patient, provides the compound described in the specification. Pharmaceutically acceptable salts include those derived from pharmaceutically acceptable inorganic or organic bases and acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid and the like. Suitable salts include those derived from alkali metals such as potassium and sodium, alkaline earth metals such as calcium and magnesium, among numerous other acids well known in the art.

Pharmaceutically acceptable “prodrugs” refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The term “heterocyclic” or “heterocycle” refers to a nonaromatic cyclic group that may be partially (contains at least one double bond) or fully saturated and wherein there is at least one heteroatom, such as oxygen, sulfur, nitrogen, or phosphorus in the ring, and wherein said “heterocyclic” or “heterocycle” group can be optionally substituted with one or more substituent selected from the group consisting of halogen, haloalkyl, alkyl, alkoxy, hydroxy, carboxyl derivatives, amido, hydroxyl, acyl, amino, alkylamino, dialkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis,” John Wiley and Sons, Second Edition, 1991, hereby incorporated by reference in its entirety. “Substituted heterocycle” is heterocycle having one or more side chains formed from non-interfering substituents.

The term “heteroaryl” or “heteroaromatic”, as used herein, refers to an aromatic that includes at least one sulfur, oxygen, nitrogen or phosphorus in the aromatic ring. Nonlimiting examples of heterocyclics and heteroaromatics are pyrrolidinyl, tetrahydrofuryl, piperazinyl, piperidinyl, morpholino, thiomorpholino, tetrahydropyranyl, imidazolyl, pyrrolinyl, pyrazolinyl, indolinyl, dioxolanyl, or 1,4-dioxanyl, aziridinyl, furyl, furanyl, pyridyl, pyrimidinyl, benzoxazolyl, 1,2,4-oxadiazolyl, 1,3,4-oxadiazolyl, 1,3,4-thiadiazole, indazolyl, 1,3,5-triazinyl, thienyl, tetrazolyl, benzofuranyl, quinolyl, isoquinolyl, benzothienyl, isobenzofuryl, indolyl, isoindolyl, benzimidazolyl, purine, carbazolyl, oxazolyl, thiazolyl, benzothiazolyl, isothiazolyl, 1,2,4-thiadiazolyl, isooxazolyl, pyrrolyl, quinazolinyl, cinnolinyl, phthalazinyl, xanthinyl, hypoxanthinyl, pyrazole, 1,2,3-triazole, 1,2,4-triazole, 1,2,3-oxadiazole, thiazine, pyridazine, benzothiophenyl, isopyrrole, thiophene, pyrazine, or pteridinyl wherein said heteroaryl or heterocyclic group can be optionally substituted with one or more substituent selected from the group consisting of halogen, haloalkyl, alkyl, alkoxy, hydroxy, carboxyl derivatives, amido, hydroxyl, acyl, amino, alkylamino, dialkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis,” John Wiley and Sons, Second Edition, 1991, hereby incorporated by reference in its entirety.

“Substituted heteroaryl” is heteroaryl having one or more non-interfering groups as substituents.

The present invention relates to compounds with a Pt(II) metal center and a plurality of S-containing ligands as anticancer agents, wherein said compounds having a general formula as the following Formula I:

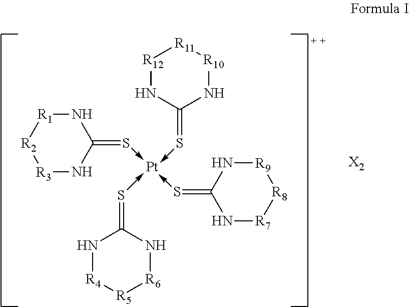

or a pharmaceutically acceptable salt, ester or prodrug thereof;

wherein:

X is selected from hydroxide, hydride, fluoride, chloride, bromide, iodide, cyanide, amide, cyanate, thiocyanate, permanganate, acetate, formate, nitrite, nitrate, hydrogen sulfate, dihydrogen phosphate, perchlorate, iodate, chlorate, bromate, chlorite, hypochlorite, hypobromite, bicarbonate or other pharmaceutically acceptable anion; and

R₁, R₂, R₃, R₄, R₅, R₆, R₇, R₈, R₉, R₁₀, R₁₁ and R₁₂ are radicals, each independently selected from substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl, preferably —CH₂—, —CH₂CH₃—.

A preferred embodiment of the compound may be tetrakis(1,3-diazinane-2-thione)platinum(II) chloride monohydrate complex ([Pt(Diaz)₄]Cl₂.H₂O of a heterocyclic thione, wherein Diaz=1,3-diazinane-2-thione), referred to as compound 1 throughout the present disclosure having the following Formula II:

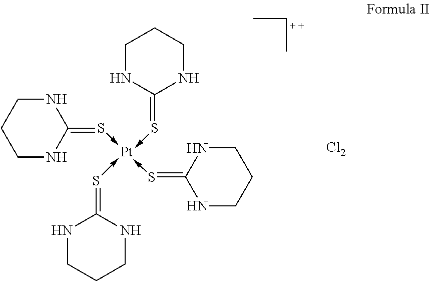

or a pharmaceutically acceptable salt, ester or prodrug thereof.

The compounds described herein can be prepared in a variety of ways known to one skilled in the art of organic synthesis or variations thereon as appreciated by those skilled in the art. The compounds described herein can be prepared from readily available starting materials. Optimum reaction conditions may vary with the particular reactants or solvents used, but such conditions can be determined by one skilled in the art.

Variations on compound 1 include the addition, subtraction, or movement of the various constituents as described for each compound. Similarly, when one or more chiral centers is present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Greene, et al., “Protective Groups in Organic Synthesis”, 2d. Ed., Wiley & Sons, 1991, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The methods described herein include a method of treating or reducing the risk of metastatic cancer. Examples of metastatic cancer include but are not limited to lung (small and non-small cell), breast, colorectal, cervical, ovarian, testicular, pancreatic, gastric, liver, bladder, head and neck, brain, oral, leukemia, gliomas, glioblastomas, sarcomas, carcinomas, lymphomas, myelomas, germ cell tumors. This method includes the steps of selecting a subject diagnosed with or at risk of developing cancer and administering to the subject an effective amount of compound 1 of derivative thereof as described herein. The compound 1 or derivative thereof as described herein can be administered systemically (e.g., orally, parenterally (e.g. intravenously), intramuscularly, intreperitoneally, transdermally (e.g., by a patch), extracorporeally, topically, by inhalation, subcutaneously or the like, or any combination thereof.

Methods can further comprise testing the efficacy of compound 1 or derivative as described herein. Testing the efficacy can include, but is not limited to, monitoring and analyzing cytotoxic activities in tumors, size of tumors. The method optionally further comprises adjusting the dosage or treatment regimen of compound 1 or derivative thereof as described herein.

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compounds described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing significant unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005, which is incorporated herein by reference in its entirety. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing compound 1 or derivative thereof as described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of compound 1 or derivative thereof as described herein include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of compound 1 or derivative thereof as described herein include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include adjuvants, such as wetting, emulsifying, suspending, sweetening, flavoring, or perfuming agents. Adjuvants include, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of compound 1 or derivative thereof as described herein for rectal administrations are preferably suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Administration of compound 1 or derivative thereof as described herein can be carried out using therapeutically effective amounts of compound 1 or derivative thereof as described herein for periods of time effective to treat metastatic cancer. The effective amount of compound 1 or derivative thereof as described herein may be determined by one of ordinary skill in the art and includes exemplary dosage amounts for a mammal of from about 0.5 to about 100 mg/kg of body weight of active compound per day, which may be administered in a single dose or in the form of individual divided doses, such as from 1 to 4 times per day. Alternatively, the dosage amount can be from about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

In these methods, the cancers being treated, e.g., lung (small and non-small cell), breast, colorectal, cervical, ovarian, testicular, pancreatic, gastric, liver, bladder, head and neck, brain, oral, leukemia, gliomas, glioblastomas, sarcomas, carcinomas, lymphomas, myelomas, germ cell tumors, can be further treated with one or more additional agents. The one or more additional agents and compound 1 or derivative thereof as described herein can be administered in any order, including simultaneous administration, as well as temporally spaced order of up to several days apart. The methods may also include more than a single administration of the one or more additional agents and/or compound 1 or derivative thereof as described herein. The administration of the one or more additional agent and compound 1 or derivative thereof as described herein may be by the same or different routes and concurrently or sequentially. When treating with one or more additional agents, compound 1 or derivative thereof as described herein can be combined into a pharmaceutical composition with the one or more additional agents. For example, compound 1 or derivative thereof as described herein can be combined into a pharmaceutical composition with another cytotoxic anticancer agent, such as, for example Rituximab, Bevacizumab (Avastin), Herceptin, Imatinib (Gleevec), Alimta, Erbitux, Velcade, Xeloda and/or Tarceva. As a further example, compound 1 or derivative thereof as described herein can be combined into a pharmaceutical composition with another platinum-based antineoplastic drug, such as, for example cisplatin, carboplatin, oxaliplatin, satraplatin, picplatin, Nedaplatin, Triplatin and Lipoplatin.

The examples below are intended to further illustrate protocols for assessing the methods and compounds described herein, and are not intended to limit the scope of the claims.

### Example 1

### Design, Synthesis and Crystal Structure of Compound 1

X-ray crystallographic and structural studies of Pt(II) complexes with S donor atom containing ligands confirm the square planar geometry around the metal center of Pt(II) complexes (L. Fuks, N. Sadlej-Sosnowska, K. Samochocka, W. Starosta, Experimental and quantum chemical studies of structure and vibrational spectra of platinum(II) and palladium(II) thiourea chlorides, J. Mol. Struct. 740 (2005) 229-235; P. J. M. W. L. Birker, J. Reedijk, G. C. Verschoor, J. Jordanov, Tetrakis(1-methyl-4-imidazoline-2-thione)platinum(II) dichloride dihydrate, Acta Crystallogr Sect. B 38 (1982) 2245-2247; Z. Popovic, D. M. Calogovic, G. Pavlovic, Z. Soldin, G. Giester, M. Rajic, D. V. Topic, Preparation, thermal analysis and spectral characterization of the 1:1 complexes of mercury(II) halides and pseudohalides with 3,4,5,6-tetrahydropyrimidine-2-thione. Crystal structures of bis(3,4,5,6-tetrahydropyrimidine-2-thione-S) mercury(II) tetrachloro and tetrabromomercurate(II), Croat. Chem. Acta 74 (2001) 359-380—each incorporated herein by reference in its entirety).

In one embodiment, the present invention discloses with the synthesis, crystal structure and anticancer activity of a new platinum (II) complex [Pt(Diaz)₄]Cl₂.H2O of the heterocyclic thione, 1-3-diazinane-2-thione (Diaz) having the following Formula III:

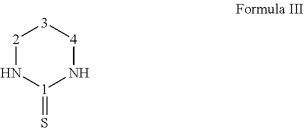

In one embodiment, solid state Pt NMR is disclosed along with other spectroscopic data of the complex.

X-ray quality single crystals are mounted in a thin-walled glass capillary on a BrukerAxs Smart Apex diffractometer equipped with a graphite monochromatized Mo Kα radiation (λ=0.71073 Å). The data are collected using SMART (SMART APEX Software (5.05) for SMART APEX detector, BrukerAxs Inc. Madison, Wis., USA, 2007—incorporated herein by reference in its entirety). The data integration may be performed using SAINT (SAINT Software (5.0) for SMART APEX Detector, BrukerAxs Inc. Madison, Wis., USA, 2007—incorporated herein by reference in its entirety). An empirical absorption correction is carried out using SADABS (G. M. Sheldrick, SADABS. Program for Empirical Absorption Correction of Area Detector Data, University of Gottingen, Germany, 1996—incorporated herein by reference in its entirety). The structure is solved with the direct methods and refined by full matrix least square methods based on F2, using the structure determination package SHELXTL (G. M. Sheldrick, SHELXTL V5.1 Software, Bruker AXS, Inc., Madison, Wis., USA, 1997 2007—incorporated herein by reference in its entirety) based on SHELX 97 (G. M. Sheldrick, A short history of SHELX, Acta Cryst. A64 (2008) 112—incorporated herein by reference in its entirety). Graphics may be generated using ORTEP-3 (L. J. Farrugia, ORTEP-3 for Windows, J. Appl. Crystallogr. 30 (1997) 565—incorporated herein by reference in its entirety) and MERCURY (C. F. Macrae, I. J. Bruno, J. A. Chisholm, P. R. Edgington, P. McCabe, E. Pidcock, L. Rodriguez-Monge, R. Taylor, J. van de Streek, P. A. Wood, Mercury CSD 2.0—new features for the visualization and investigation of crystal structures, J. Appl. Crystallogr. 41 (2008) 466-470—incorporated herein by reference in its entirety). Nitrogen atoms belonging to the NH groups are assumed to have sp² hybridization. For compound 1, one ligand carbon atom (C3) presents a two-site (C3A, C3B) disorder, the main component may be refined to a site occupancy of 0.66(2). H-atoms of the water molecule are located on a Fourier difference map and refined isotropically. Table 1 depicts the crystallographic data (crystal and structure refinement) for compound 1, while Tables 2 and 3 show the selected bond lengths (Å) and angles (°) for compound 1, respectively.

TABLE 1
Crystal and structure refinement data for compound 1.

| Parameter | Value |
|---|---|
| CCDC deposit no. | 857990 |
| Empirical formula | C₁₆H₃₄Cl₂N₈OPtS₄ |
| Formula weight | 748.74 |
| Crystal size/mm | 0.58 × 0.17 × 0.15 |
| Wavelength/Å | 0.71073 |
| Temperature/K | 301(2) |
| Crystal symmetry | Monoclinic |
| Space group | P2₁/c |
| Unit cell dimensions | |
| a/Å | 8.5945(4) |
| b/Å | 14.2550(7) |
| c/Å | 23.3451 |
| β/° | 91.3831 |
| Volume (Å³) | 2859.2(2) |
| Z | 4 |
| Calc. density (g cm⁻³) | 1.739 |
| μ(Mo-Kα)/mm⁻¹ | 5.411 |
| F(000) | 1480 |
| θ range (°) | 1.67-28.29 |
| Limiting indices | −11 ≦ h ≦ 11 |
| | −19 ≦ k ≦ 19 |
| | −31 ≦ l ≦ 31 |
| Max and min transmission | Tₘᵢₙ = 0.1454, Tₘₐₓ = 0.4974 |
| Data/restraints/parameters | 7104/44/308 |
| Goodness-of-fit on F² | 1.040 |
| Final R indices [I > 2σ(1)] | R₁ = 0.0236, wR₂ = 0.0538 |

TABLE 2
Selected bond lengths (Å) for compound 1.

| Bond | Bond length (Å) |
|---|---|
| Pt1-S1 | 2.3240(8) |
| Pt1-S2 | 2.3321(8) |
| Pt1-S3 | 2.3111(8) |
| Pt1-S4 | 2.3263(8) |

TABLE 3
Selected bond angles (°) for compound 1.

| Bond | Bond angle (°) |
|---|---|
| S1-Pt1-S2 | 93.67(3) |
| S1-Pt1-S4 | 88.01(3) |
| S3-Pt1-S1 | 171.53(3) |
| S3-Pt1-S4 | 87.99(3) |
| S3-Pt1-S2 | 89.25(3) |
| S4-Pt1-S2 | 171.72(3) |

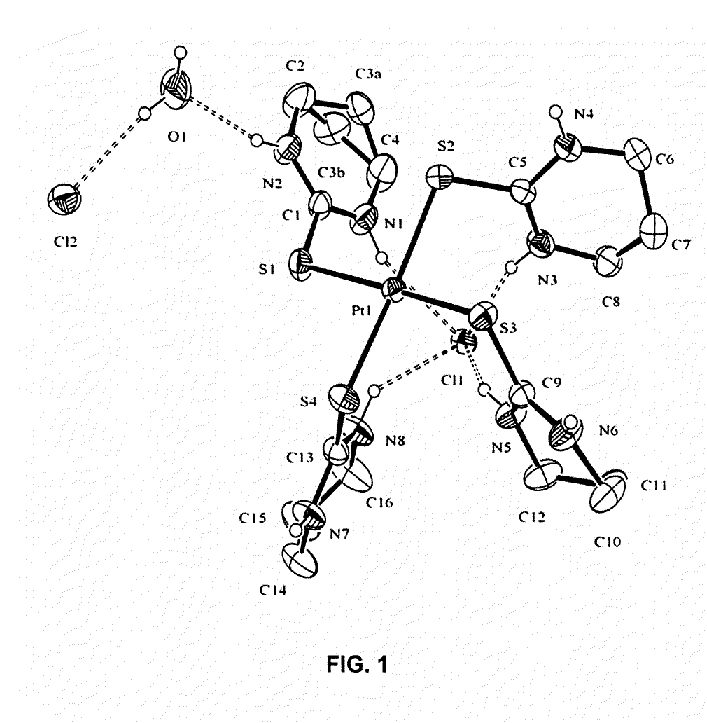

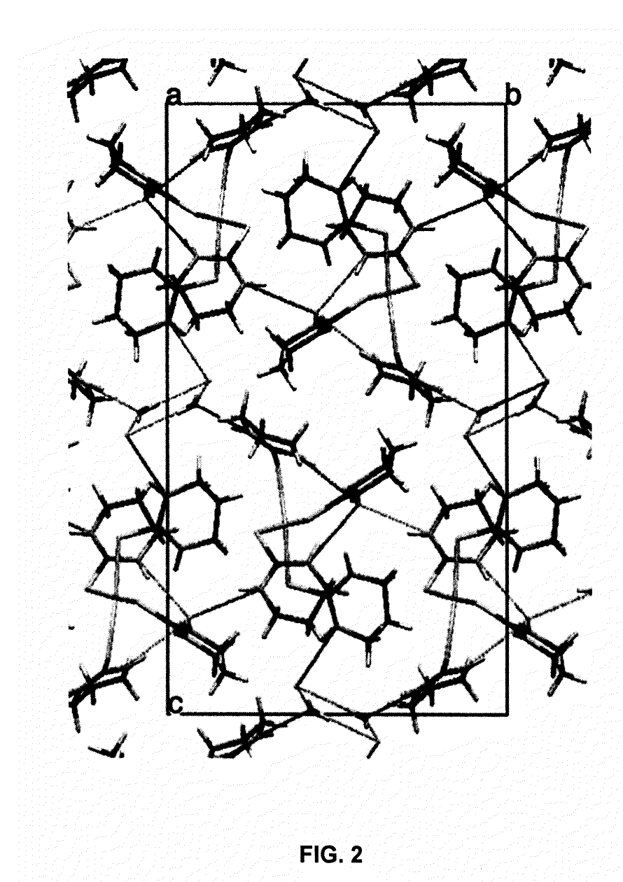

The X-ray structure of compound 1 is shown in FIG. 1. As can be seen from the structure, Pt(II) ion is bonded to four sulfur atoms, each belonging to a Diaz ligand. The Pt—S bond lengths are in the range 2.3111(8)-2.3321(8) Å, while the S—Pt—S bond angles are in the range 87.99(3)-93.67(3)°. These values are similar to those found for tetrakis(thiourea-S)-platinum(II) chloride (L. Fuks, N. Sadlej-Sosnowska, K. Samochocka, W. Starosta, Experimental and quantum chemical studies of structure and vibrational spectra of platinum(II) and palladium(II) thiourea chlorides, J. Mol. Struct. 740 (2005) 229-235—incorporated herein by reference in its entirety) and tetrakis(1-methyl-4-imidazoline-2-thione)-platinum(II) chloride dihydrate (P. J. M. W. L. Birker, J. Reedijk, G. C. Verschoor, J. Jordanov, Tetrakis(1-methyl-4-imidazoline-2-thione)platinum(II) dichloride dihydrate, Acta Crystallogr Sect. B 38 (1982) 2245-2247—incorporated herein by reference in its entirety). The SCN₂ moieties of the four ligand molecules are essentially planar with the S—C and C—N bond lengths in the ranges (1.722(3)-1.744(3) Å) and (1.304(4)-1.324(4) Å) respectively. The corresponding bond lengths previously reported for the free ligand are d_{(S-C)}=1.720 Å and d_{(C-N)}=1.331 (Å Z. Popovic, D. M. Calogovic, G. Pavlovic, Z. Soldin, G. Giester, M. Rajic, D. V. Topic, Preparation, thermal analysis and spectral characterization of the 1:1 complexes of mercury(II) halides and pseudohalides with 3,4,5,6-tetrahydropyrimidine-2-thione. Crystal structures of bis(3,4,5,6-tetrahydropyrimidine-2-thione-S) mercury(II) tetrachloro and tetrabromomercurate(II), Croat. Chem. Acta 74 (2001) 359-380—incorporated herein by reference in its entirety). The significantly longer S—C bond distances, associated with shorter C—N bonds in the complex, are consistent with significant C—N double bond character and electron donation from the ligand to the metal ion. Each of the four Diaz ligands is engaged in hydrogen bonding interactions with one chloride counter ion (Cl1). This results in an umbrella type structure where all tetrahydropyrimidine rings are on the same side of the PtS₄ mean plane, as shown in FIG. 1). Referring to FIG. 2, other hydrogen bonding interactions also take place including those with a water molecule present in the lattice.

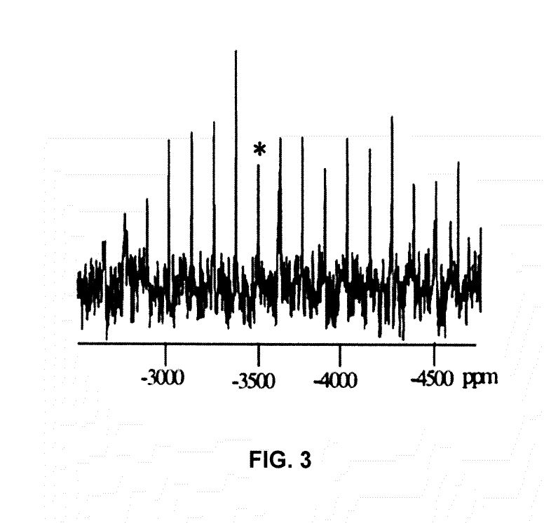

In one embodiment, ¹H NMR spectra may be obtained on JEOL JNM-LA 500 NMR spectrometer operating at a frequency of 500.00 MHz. ¹³C NMR spectra may be obtained at the frequency of 125.65 MHz with ¹H broadband decoupling at 298 K. The spectral conditions are: 32 k data points, 0.967 s acquisition time, 1.00 or 30.00 s pulse delay and 45° pulse angle. Solid state cross-polarization magic-angle spinning (CPMAS)¹⁹⁵Pt{¹H} NMR spectrum of compound 1 may be obtained at ambient temperature on a Bruker 400 NMR spectrometer operating at a frequency of 85.94 MHz. Contact time of 3 ms may be used with a recycle delay of 10 s. Approximately 5000 FIDs are collected and transformed with a line broadening of 100 Hz. Chemical shifts may be referenced using an external sample of solid K₂[PtCl₆]. The sample may be spun at two speeds, 11 and 8 kHz, at the magic angle to identify the center peak. The CPMAS spectra containing spinning side-band manifolds may be analyzed using a computer software WSOLIDS developed at Dalhousie and Turbingen universities, to yield the anisotropy and asymmetry parameters (K. Eichele, R. E. Wasylischen, W: Simulation Package, Version 1. 4. 4, Dalhousie University, Halifax, Canada; University of Tubingen, Tübingen, Germany, 2001—incorporated herein by reference in its entirety). The spectrum obtained at 11 kHz is shown in FIG. 3 and the NMR parameters are as follows: isotropic chemical shift=−3500 ppm; anisotropy=2196 ppm and asymmetry=0.71. The ¹³C NMR chemical shifts of the free thione ligand and its complex are studied in 50:50 (v/v) mixtures of CDCl₃ and DMSO-d₆. The NMR data are given in Table 4.

TABLE 4
¹³C chemical shifts (ppm) of the thione and Pt(II)-thione
compound 1 in 50:50 (v/v) mixture of CDCl₃ and DMSO-d₆.

| Species | N—H | C-1 | C-2 | C-3 | C-4 |
|---|---|---|---|---|---|
| Diaz | 6.75 | 176.81 | 40.59 | 19.3 | 40.59 |
| 1 | 9.08 | 167.68 | 40.15 | 18.86 | 40.15 |

The N—H protons of the coordinated thione are shifted toward high frequency with respect to the free thione. This large de-shielding of the N—H protons is an indication of the increase in the double bond character of the C—N bond upon coordination to Pt(II), which is consistent with the coordination of thiourea or its derivatives to the metal via the sulfur atom (P. Castan, J. Laurent, Platinum(II) complexes with ligands involving the —NH—CS—NH— group in heterocyclic rings, Transit. Met. Chem. 5 (1980) 154-157; A. A. Isab, M. I. M. Wazeer, Complexation of Zn(II), Cd(II) and Hg(II) with thiourea and selenourea: 1H, 13C, 15N, 77Se and 113Cd solution and solid-state NMR study, J. Coord. Chem. 58 (2005) 529-537; A. A. Isab, S. Ahmad, M. Arab, Synthesis of silver(I) complexes of thiones and their characterization by ¹³C, ¹⁵N and ¹⁰⁷Ag NMR, Polyhedron 21 (2002) 1267-1271—each incorporated herein by reference in its entirety). The appearance of a N\H signal is an evidence of coordination of Pt(II) via the thione group. On the other hand, the ¹³C NMR signal for the thiocarbonyl carbon in the complex shifted upfield by 9.13 ppm with respect to the free ligand. This shift in thiocarbonyl carbon and NH proton signals is attributed to the reduction in C—S bond order and an increase in C—N bond order upon complexation (P. Castan, J. Laurent, Platinum(II) complexes with ligands involving the —NH—CS—NH— group in heterocyclic rings, Transit. Met. Chem. 5 (1980) 154-157; A. A. Isab, M. I. M. Wazeer, Complexation of Zn(II), Cd(II) and Hg(II) with thiourea and selenourea: 1H, 13C, 15N, 77Se and 113Cd solution and solid-state NMR study, J. Coord. Chem. 58 (2005) 529-537—each incorporated herein by reference in its entirety).

The solid state ¹⁵N and ¹³C NMR data, shown in Table 5, indicate that the complexation of Pt(II) with the thione resulted in shielding of thiocarbonyl carbons in compound 1 by about 8 ppm in comparison with their free thione ligand (M. I. M. Wazeer, A. A. Isab, A. El-Rayyes, Solid-state NMR study of 1,3-imidazolidine-2-thione, 1,3-imidazolidine-2-slenone and some of their N-substituted derivatives, Spectroscopy 18 (2004) 113-119—incorporated herein by reference in its entirety). This observation confirms that the thione form is retained in the compound 1. Furthermore, three different environments for nitrogen atoms are observed in compound 1, which is about 5 to 8 ppm de-shielded in comparison with the free ligand. Such three different environments for nitrogen are consistent with the X-ray structure, revealing three hydrogen bonding schemes for the N—H groups namely N—H—Cl, N—H—S and N—H—O.

TABLE 5
¹⁵N and ¹³C solid NMR chemical shifts (ppm)
for the ligands and their Pt compound 1.

| Compound | N-1 | C-1 | C-2 | C-3 | C-4 |
|---|---|---|---|---|---|
| Diaz | −273.43 | 175.22 | 39.79 | 19.9 | 39.79 |
| 1 | −265.15^{a} | 168.38 | 42.39 | 20.96 | 42.39 |
| | −270.26^{a} | 167.38 | 41.6 | 19.91 | 41.6 |
| | −274.87^{a} | 166.41 | | | |

The potassium tetrachloroplatinate(II) is reported to have an axial symmetry, and the tensor has a very large chemical shift anisotropy of 10,414 ppm (M. I. M. Wazeer, A. A. Isab, A. El-Rayyes, Solid-state NMR study of 1,3-imidazolidine-2-thione, 1,3-imidazolidine-2-slenone and some of their N-substituted derivatives, Spectroscopy 18 (2004) 113-119—incorporated herein by reference in its entirety). The ¹⁹⁵Pt NMR chemical shift of compound 1 lies in the same range observed for other Pt(II) complexes surrounded by four sulfur containing ligands in square planar geometry (S. W. Sparks, P. D. Ellis, Platinum-195 shielding tensors in potassium hexachloroplatinate(IV) and potassium tetrachloroplatinate(II) J, Am. Chem. Soc. 108 (1986) 3215-3218; J. D. Woollins, A. Woollins, B. Rosenberg, Detection of trace amounts of trans-[Pt(NH₃)₂Cl₂] in the presence of cis-[Pt(NH₃)₂Cl₂]. A high performance liquid chromatographic application of Kurnakow's test, Polyhedron 2 (1983) 175-178—each incorporated herein by reference in its entirety). The observed lower anisotropy in our complex may be attributed to the deviation from the perfect axial symmetry as shown in FIG. 3.

The solid-state mid-IR spectra of the ligand and tetrakis(1,3-diazinane-2-thione)platinum(II) chloride monohydrate compound 1 may be recorded on a Perkin-Elmer FTIR 180 spectrophotometer using KBr pellets over the range 4000-400 cm⁻¹. The selected mid-IR frequencies are given in Table 6. The presence of ν(C═S), ν(N—H) and ν(C—N) absorption bands validates the coordination of ligand to Pt(II) ion in the solid state (P. D. Akrivos, Recent studies in the coordination chemistry of hetero-cyclicthiones and thionates, Coord. Chem. Rev. 213 (2001) 181-210; M. R. Malik, V. Vasylyeva, K. Merz, N. Metzler-Nolte, M. Saleem, S. Ali, A. A. Isab, K. S., Munawar, S. Ahmad, Synthesis, crystal structures, antimicrobial properties and enzyme inhibition studies of zinc(II) complexes of thiones, Inorg. Chim. Acta 376 (2011) 207-211; S. Ahmad, Q. Amir, G. Naz, A. Fazal, M. Fettouhi, A. A. Isab, T. Rüffer, H. Lang, Synthesis and crystal structures of cadmium iodide complexes of N,N-diethylthiourea and 1,3-diazinane-2-thione, J. Chem. Crystallogr. 42 (2012) 615-620; A. A. Seerat-ur-Rehman, M. N. Isab, T. Tahir, M. Khalid, H. Saleem, S. Sadaf, Ahmad, Synthesis, crystal structure and antimicrobial studies of a thione derivative of transplatin, trans-[Pt(NH₃)₂(Diaz)₂]Cl₂.2H2O (Diaz=1,3-diazinane-2-thione), Polyhedron 36 (2013) 68-71—each incorporated herein by reference in its entirety).

TABLE 6
Selected mid-IR frequencies (cm⁻¹)
of free ligand and its Pt(II) compound 1.

| | IR frequencies | | |
|---|---|---|---|
| Compound | ν(C═S) | ν(C—N) | ν(N—H) |
| Diaz | 512 | 1455 | 3250 |
| 1 | 503 | 1581 | 3185, 3260, 3310 |

### Example 2

### Cytotoxicity Evaluations of Compound 1

Anticancer activity tests were evaluated for compound 1 and cisplatin against four human cancers namely A549 (human lung carcinoma), MCF7 (human breast cancer), HCT15 (human colon adenocarcinoma) and HeLa (human cervical cancer) cell lines. The dose dependent in vitro cytotoxic effect was obtained by the stipulated increase in concentrations of compound 1 and cisplatin against the fixed number of human cancer cells. The IC₅₀ concentrations of compound 1 and cisplatin for different human cell lines were obtained from curves between complex concentration and percentage viability of cells. The IC₅₀ values of the compound 1 ranged far and wide between 19.1 and 93.1 μM (Table 7).

TABLE 7
IC₅₀ values (μM) of Pt(II) compounds against
A549, MCF7, HCT15 and HeLa cancer cell lines.

| Compound | A549 | MCF7 | HCT15 | HeLa |
|---|---|---|---|---|
| Cisplatin | 41.6 | 22.4 | 29.5 | 19.4 |
| 1 | 56.4 | 19.1 | 47.1 | 93.1 |

In the present examples, the IC₅₀ values for cisplatin and compound 1 against A549 are 41.6 and 56.4 μM respectively. For this reason, the in vitro cytotoxicity of compound 1 in terms of IC₅₀ values against A549 cell line is somewhat higher than that for cisplatin. As a result, compound 1 is not as good a cytotoxic agent as cisplatin. The in vitro cytotoxicity of compounds cisplatin and 1, in terms of IC₅₀ values against MCF7 cell line are found to be 22.4 and 19.1 μM respectively. As far as, in vitro cytotoxicity against MCF7 cell line is concerned, compound 1 is a reasonably better cytotoxic agent than cisplatin. The in vitro cytotoxicity of compounds 1 and cisplatin in terms of IC₅₀ values against HCT15 cell line are 47.1 and 29.5 μM respectively. Therefore, cisplatin is 1.6 times better cytotoxic agent than compound 1. The cytotoxicity of compounds 1 and cisplatin in terms of IC₅₀ values against HeLa cell line is found to be 93.1 and 19.4 respectively. The compound 1 with an IC₅₀ value, i.e. 93.1 is a lesser cytotoxic candidate than cisplatin.

In view of the foregoing paragraph, compound 1 is found to be most effective against MCF7 (human breast cancer) cell line with an IC₅₀ value of 19.1 μM and indicates better cytotoxicity than cisplatin. The cytotoxicity against the rest of cancer cell lines is lesser than that of cisplatin, but within micrometer range. Disclosed embodiments show the selective cytotoxicity of compound 1 against a particular cancer cell line. In drug design, selective activity of the drug molecule is crucial. This anticancer activity study suggests that compound 1 possesses the tendency of further ligand exchange with biomolecules like proteins and DNA (A. A. Seerat-ur-Rehman, M. N. Isab, T. Tahir, M. Khalid, H. Saleem, S. Sadaf, Ahmad, Synthesis, crystal structure and antimicrobial studies of a thione derivative of transplatin, trans-[Pt(NH₃)₂(Diaz)₂]Cl₂.2H₂O (Diaz=1,3-diazinane-2-thione), Polyhedron 36 (2013) 68-71; D. Kovala-Demertzi, M. A. Demertzis, J. R. Miller, C. Papadopoulou, C. Dodorou, G. Filousis, Platinum(II) complexes with 2-acetyl pyridine thiosemicarbazone: synthesis, crystal structure, spectral properties, antimicrobial and antitumour activity, J. Inorg. Biochem. 86 (2001) 555-563; S. Ahmad, A. A. Isab, S. Ali, A. R. Al-Arfaj, Perspectives in bioinorganic chemistry of some metal based therapeutic agents, Polyhedron 25 (2006) 1631-1643—each incorporated herein by reference in its entirety).

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1-2. (canceled)

3: A pharmaceutical composition comprising a compound of Formula I or a pharmaceutically acceptable salt, ester or prodrug thereof, and at least one pharmaceutically acceptable carrier;
wherein Formula I is: 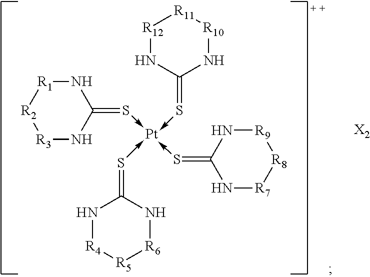
wherein:
X is selected from hydroxide, hydride, fluoride, chloride, bromide, iodide, cyanide, amide, cyanate, thiocyanate, permanganate, acetate, formate, nitrite, nitrate, hydrogen sulfate, dihydrogen phosphate, perchlorate, iodate, chlorate, bromate, chlorite, hypochlorite, hypobromite and bicarbonate; R₁-R₁₂ are each a C₁ alkylene; and
the at least one pharmaceutically acceptable carrier is selected from the group consisting of, isopropyl alcohol, ethyl carbonate, ethyl acetate, benzyl alcohol, benzyl benzoate, propyleneglycol, 1,3-butyleneglycol, dimethylformamide, an oil, a polyol, an injectable organic ester, glycerol, tetrahydrofurfuryl alcohol, a fatty acid ester of sorbitan, cocoa butter, a suppository wax, an amino acid, a carbohydrate, a sugar alcohol, a low molecular weight polypeptide having 10 residues or less, and a protein.

4: The pharmaceutical composition of claim 3, wherein the compound of Formula I is: 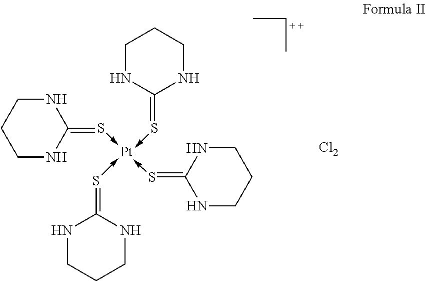
or a pharmaceutically acceptable salt, ester or prodrug thereof.

5-6. (canceled)

7: The pharmaceutical composition of claim 3, further comprising one or more non-active ingredients.

8: The pharmaceutical composition of claim 3, wherein the pharmaceutical composition is in solid, semi-solid or liquid dosage forms.

9: The pharmaceutical composition of claim 3, wherein the pharmaceutical composition is administered to a subject in need thereof systemically, parenterally, intramuscularly, intraperitoneally, transdermally, extracorporeally, topically, or any combination thereof.

10-21. (canceled)
